(19) United States
(12) Patent Application Publication
(10) Pub. No.: US 2005/0130887 A1
(43) Pub. Date: Jun. 16, 2005

(54) Genetic sequence related to bone diseases

(76) Inventors: Jean Vacher, Quebec (CA); Nader Chalhoub, Quebec (CA); Nadia Benachenou, Quebec (CA)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/491,215

(22) PCT Filed: Sep. 25, 2002
(86) PCT No.: PCT/EP02/10721

(30) Foreign Application Priority Data
Sep. 27, 2001 (CA) 2357987

(51) Int. Cl. A61K 38/17; A61K 48/00

(52) U.S. Cl. 514/12; 514/44

(57) ABSTRACT

The identification, isolation and cloning of gl gene which when mutated is associated with bone related diseases as well as its transcript in gene products. A method of diagnostic and detection of potential carriers of this mutated gene, bone related diseases, diagnosis, gene therapy recombinant technology and therapy using the information derived from the DNA, protein and the function of the protein is also provided.

### BACKGROUND OF THE INVENTION

1. Field of the Invention

The present invention generally relates to the field of bone related diseases associated with osteoclast cells dysfunction. More particularly, the invention is concerned with the identification, isolation and cloning of a gene, which when mutated is associated with bone related diseases as well as its transcript in gene products. The present invention also relates to a method of diagnostic and detection of potential carriers of this mutated gene, bone related diseases, diagnosis, gene therapy recombinant technology and therapy using the information derived from the DNA, protein and the function of the protein.

A) Brief Description of the Prior Art

Bone homeostasis is dependent on two opposite and dynamic processes of bone formation and resorption in vertebrates and is regulated throughout adult life. Defective bone resorption (osteopetrosis or osteoporosis) results from a defect in bone resorption. More particularly, osteopetrosis results in accumulation of mineralised bone and cartilage due to a lack of bone remodelling activity. This activity is normally provided by osteoclast. Such fully differentiated cells are multinucleated and are formed by the fusion of myeloid cells from the monocyte-macrophage lineage.

Osteopetrosis results from a defect in the differentiation or the activation of the osteoclast, a specialized cell, which derives from the granulocyte-macrophage hematopoietic lineage. The role of the osteoclast is bone tissue resorption, a process that is counterbalanced by the osteoblast activity that results in bone tissue formation. When such balance is disrupted, major bone diseases as osteoporosis and osteopetrosis can occur. Lazner, F. et al., Hum Mol Genet., 8:1839-1846 (1999).

The event of homologous recombination in association with gene targeting in the mouse, tremendously improved our understanding of osteoclastogenesis. The specific loss of osteoclast gene function resulted in osteopetrosis that is characterized by a general increase in bone mass. For example, PU-1, c-fos, NFk-B and RANKL gene activities are required for the differentiation/proliferation of osteoclast precursors, while the loss of c-src, TRAF6, V-ATPase and CIC-7 have been associated with defects in polarization/resorption of the osteoclast. Karsenty, G., Genes and Dev., 13:3037-3051 (1999); Teitelbaum, S. L., Science, 289:1504-1508 (2000).

In addition to these engineered mutations, four spontaneous mutations have been described in the mouse. The op gene encodes the hematopoietic colony stimulating factor 1 (CSF-1) Yoshida, H. et al., Nature, 345:442-445, (1990), mi encodes a transcriptional factor from the basic-loop-helix zipper (bHLH-zip) family, Hodgkinson, C. A., Cell, 74:395404, (1993) and the oc mutation affects the 116KD subunit of the V-ATPase (Scimeca, J-C et al., Bone, 26:207-213 (2000). The fourth mutation, grey-lethal (gl), described for the first time by Grunberg, Gruneberg, H. J. Hered., 27:107-109 (1936), displays an osteopetrotic phenotype closely related to the most severe autosomal recessive form of the human disease. As in humans, early death occurs around three weeks of age in homozygous gl/gl mice, and functional rescue can be obtained following bone marrow transplantation demonstrating a cell-autonomous defect. Walker, D. G., Science, 190:784-785 (1975).

Therefore, there is a need to determine the nucleic acid sequence encoding for an osteoclast-related polypeptide having the biological activity of modulating the bone resorption.

The inventors have determined that the gl gene is required for osteoclast maturation/function. Rajapurohitam, V. et al., Bone, 28:513-523 (2001).

### SUMMARY OF THE INVENTION

The present invention originates from the discovery of a gl gene encoding a polypeptide involved in the regulation of bone resorption in a mammal.

Accordingly, the present invention relates to an isolated or purified nucleic acid molecule encoding a mammalian osteoclast-related polypeptide (referred to hereinafter the Gl polypeptide) having the biological activity of modulating bone resorption in osteoclast cells.

The present invention also provides the following:
an expression or cloning vector having the nucleic acid sequence of the Gl polypeptide mentioned above;
a host cell having the above mentioned expression or cloning vector;
a non-human mammal comprising a genetically modified nucleic acid molecule of the Gl polypeptide of the present invention;
an isolated antibody that binds specifically to the Gl polypeptide and fragments thereof;
a process for producing the Gl polypeptide of the present invention;
a method for preventing or treating a bone resorption-related disease in a mammal subject by administering the polypeptide of the present invention to the subject; and
a pharmaceutical composition containing the Gl polypeptide of the present invention, for preventing or treating an osteoclast-related disease such as osteoporosis and osteopetrosis.

In summary, the work conducted in the context of the present invention has allowed the inventors to identify a novel gene with a specific function that is absolutely required for proper osteoclast maturation and bone tissue resorption.

This in turn, has allowed the inventors of the present application to provide methods, pharmaceutical compositions and diagnostic tools to treat and/or prevent bone related diseases such as osteopetrosis and osteoporosis.

### BRIEF DESCRIPTION OF THE DRAWINGS

FIG. 1 shows the physical entrance cryptional map of BAC.

FIG. 2 is a table regrouping information on the characterisation of BAC clones.

FIG. 3 shows the expression of the Gl gene in different tissue type.

FIG. 4 shows the expression of the Gl gene in transgenic mice in different tissue type.

FIG. 5A shows the result of the Western blot ANALYSIS OF Gl polypeptide in Wild-type and gl osteoclasts.

FIG. 5B shows the specific cytoplasmic localisation of the Gl polypeptide. FIG. 6 shows the Kyte-Doolittle hydropathy plot for mouse GL polypeptide.

FIG. 7 shows the TMpred-prediction of transmembrane Regions and Orientation for Mouse Gl polypeptide.

FIG. 8 shows the nucleic acid sequence of the mouse Gl gene.

FIG. 9 shows the nucleic acid sequence of the human GL gene homologue.

FIG. 10 shows the amino acid sequence of the mouse Gl polypeptide.

FIG. 11 shows the amino acid sequence of the human GL polypeptide.

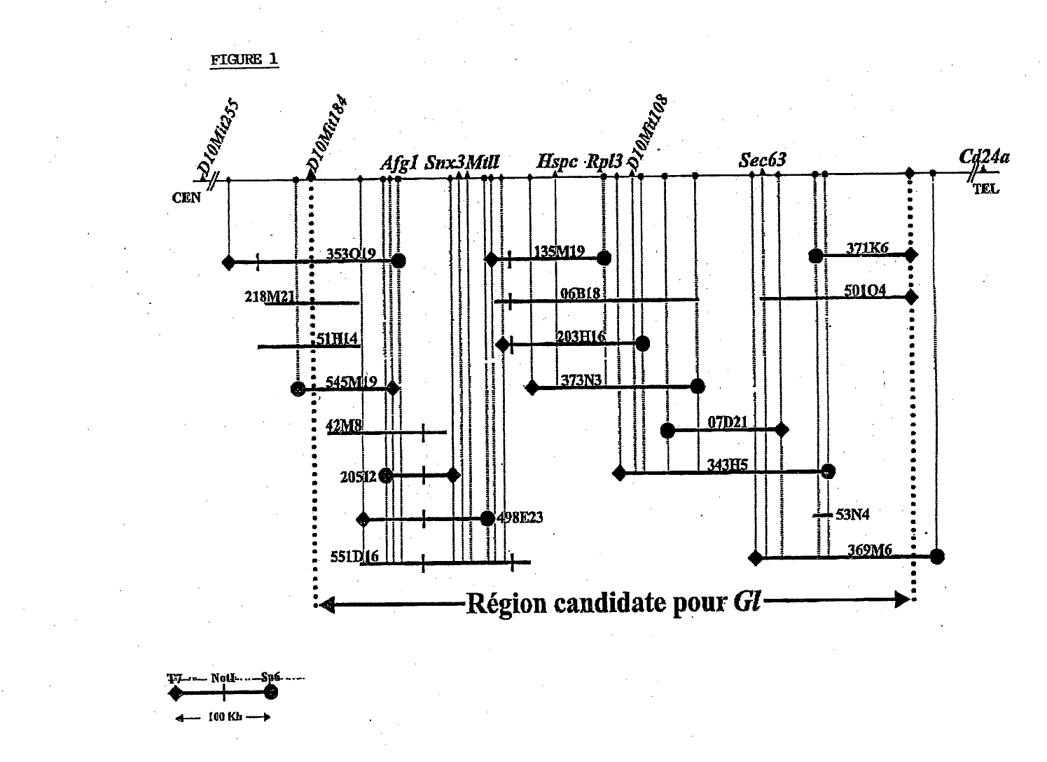

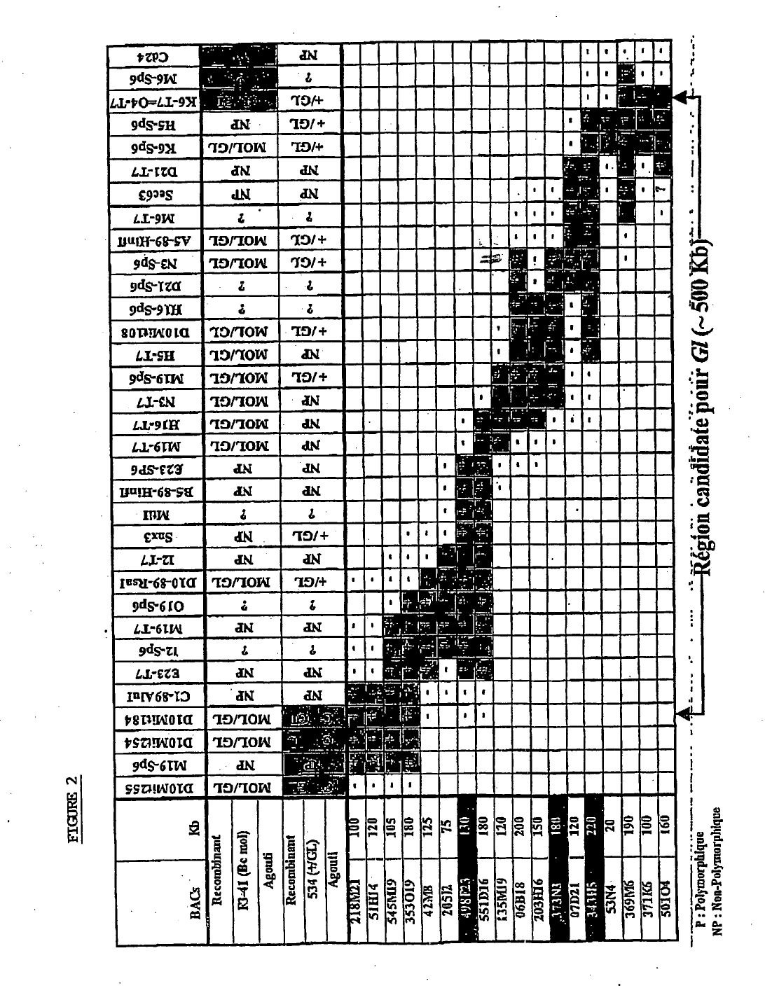

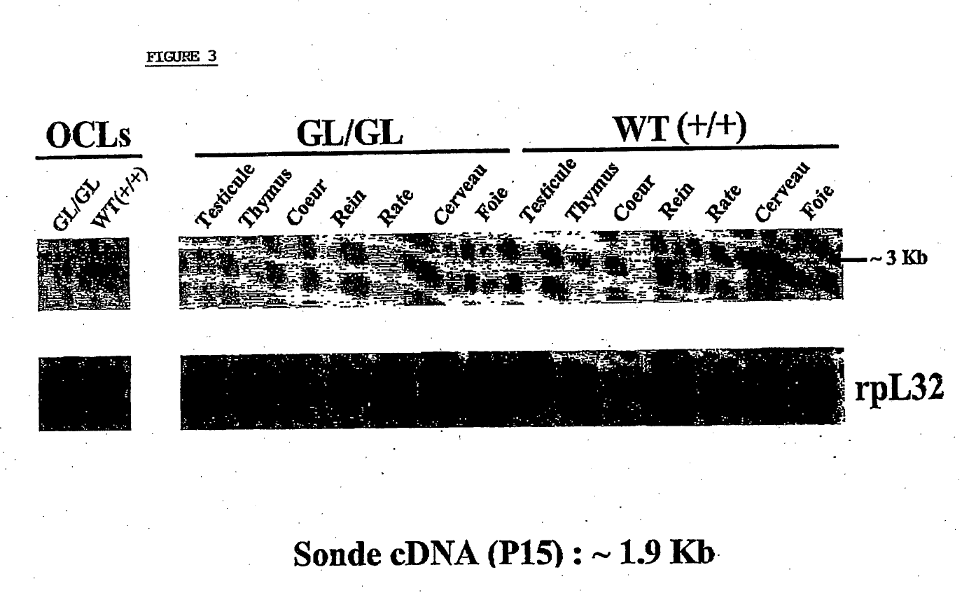

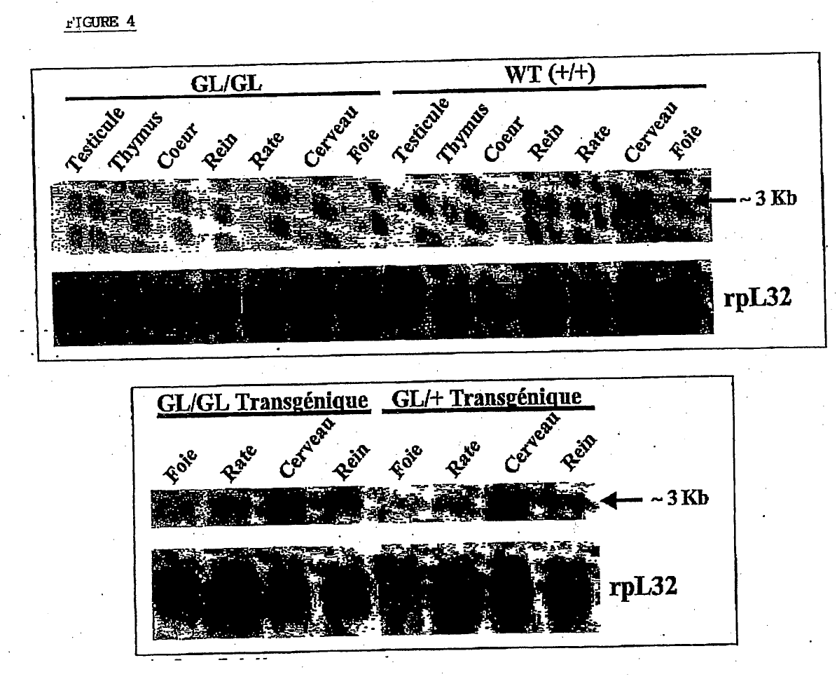

### DETAILED DESCRIPTION OF THE INVENTION

A) Definitions

In order to provide an even clearer and consistent understanding of the specification and the claims, including the scope given herein to such terms, the following definitions are provided:
Osteoclast broadly relates to a large multinucleated cell found in growing bone that resorbs bone tissue, as the renewal of bone matrix.
gl gene also relates to a gene, which encodes for an osteoclast-related polypeptide and which when mutated is associated with bone related diseases. This definition is understood to include the various sequence polymorphisms that exist wherein the codon substitutions or deletion in the gene sequence do not affect the essential function of the gene product as well as functionally equivalence of the nucleotide sequences of SEQ. ID No. 1 and SEQ. ID No. 2. This term also relates to an isolated coding sequence, but can also include some or all of the flanking regulatory elements and/or introns. The term gl gene includes the gene and other species homologous to the human gene, which when mutated is associated with bone related diseases.

Gl polypeptide refers to the polypeptide encoded by the gl gene. This polypeptide may be a natural or synthetic compound containing two or more amino acids having a specificity to osteoclast cells susceptible to modulate the activity of osteoclast cells. The preferred source of polypeptide is the mammalian polypeptide as isolated from humans or animals. The polypeptide may be produced by recombinant organisms or chemically or enzymatically synthesised. This definition is understood to include functional variance, such as the various polymorphic forms of the protein where the amino acids substitution or deletion within the amino acid sequence do not effect essential functioning of the protein or its structure. It also enclosed functional fragments of osteoclast related polypeptide.
Modulation refers to activation or inhibition of osteoclast cell activity in bone resorption.
Ruffled border broadly refers to the folded configuration of the osteoclast cell membrane through which the osteoclast may resorb the bone matrix.
Functional homologues broadly refer to a protein/peptide or polypeptide sequence that possesses a functional biological activity that is substantially similar to the biological activity of the whole protein/peptide or polypeptide sequence. A functional derivative of a protein/peptide or polypeptide may or may not contain post-translational modifications such as covalently linked carbohydrate, if such modification is not necessary for the performance of a specific function. The term “functional derivative” is intended to cover the “fragments”, “segments”, “variants”, “analogs” or “chemical derivatives” of a protein/peptide or polypeptide.
Analog broadly refers to a peptide or polypeptide that is substantially similar in function to the polypeptide of the invention.
Derived broadly refers to a protein/peptide or polypeptide that is said to “derive” from a protein/peptide or from a fragment thereof when such protein/peptide comprises at least one portion, substantially similar in its sequence, to the native protein/peptide or to a fragment thereof.
Isolated or Purified refers to a state different from the natural state. More precisely, it is altered “by the hand of man” from its natural state, i.e., if it occurs in nature, it has been changed or removed from its original environment, or both. For example, a polynucleotide naturally present in a living organism is not “isolated”, the same polynucleotide separated from the coexisting materials of its natural state, obtained by cloning, amplification and/or chemical synthesis is “isolated” as the term is employed herein. Moreover, a polynucleotide that is introduced into an organism by transformation, genetic manipulation or by any other recombinant method is “isolated” even if it is still present in said organism.
The term peptide or polypeptide herein includes any natural or synthetic compounds containing two or more amino acids. Therefore, it comprises proteins, glycoproteins, and protein fragments derived from pathogenic organisms such as viruses, bacteria, parasites and the like, or proteins isolated from normal or pathogenic tissues, such as cancerous cells. It also includes proteins and fragments thereof produced through recombinant means that has been associated or not with other peptides coding for tumoral, viral, bacterial or fungic epitopes for forming a fusion protein.
Nucleic acid broadly refers to any DNA, RNA sequence or molecule having one nucleotide or more, including nucleotide sequences encoding a complete gene. The term is intended to encompass all nucleic acids whether occurring naturally or non-naturally in a particular cell, tissue or organism. This includes DNA and fragments thereof, RNA and fragments thereof, cDNAs and fragments thereof, expressed sequence tags, artificial sequences including randomized artificial sequences.
Functional homologues broadly refer to any molecule, natural or synthetic, being able to carry out the same functions as the protein or polypeptide of interest.
The term “variant” as is generally understood and used herein, refers to a protein that is substantially similar in structure and biological activity to either the protein or fragment thereof. Thus two proteins are considered variants if they possess a common activity and may substitute each other, even if the amino acid sequence, the secondary, tertiary, or quaternary structure of one of the proteins is not identical to that found in the other.
Vector refers to a self-replicating RNA or DNA molecule, which can be used to transfer an RNA or DNA segment from one organism to another. Vectors are particularly useful for manipulating genetic constructs and different vectors may have properties particularly appropriate to express protein(s) in a recipient during cloning procedures and may comprise different selectable markers known by one skilled in the art. Bacterial plasmids are commonly used vectors.
Probe or primer broadly refers to any DNA or RNA sequence that is marked with a fluorescent compound, a radioisotope or an enzyme and used for detecting homologues (complementary) sequences as by hybridization in situ or in vitro.
Osteoporosis relates to a disease in which the bones become extremely porous, are subject to fracture, and heal slowly, occurring especially in women following menopause and often leading to curvature of the spine from vertebral collapse.
Osteopetrosis relates to a disease in which the bones become extremely dense. There is absence of development of the bone marrow, of teeth growth and of general growth. This disease also causes premature death of the subjects.
   B) Overview of the Invention

The present invention is concerned with the identification and sequencing of the mammalian gl gene in order to gain insight into the cause and etiology of bone related diseases. From this information, screening methods and therapies for the diagnosis and treatment of the diseases can be developed.

Although it is generally understood that bone related diseases are caused by osteoclast related polypeptide expressed most likely in the bones, expression of this polypeptide has been found in variety of mammalian tissue types such as the testis, the thymus, the heart, the kidney, the spleen, the brain and the liver.

The mutation identified in the context of the present invention has been related to bone diseases such as osteopetrosis and osteoporosis. With the identification of sequences of the gene and the gene products, probes and antibodies raised against the gene product can be used in a variety of hybridisation and immunological assays to screen for and detect the presence of either a normal or mutated gene or gene product.

Patient therapy through removal or blocking of the mutant gene product, as well as supplementation with the normal gene product by amplification, by genetic and recombinant techniques or by immunotherapy can now be achieved. Correction or modification of the defective gene product by protein treatment immunotherapy (for example using antibodies to the defective protein) or knock out of the mutated gene is now also possible.

The bone related disease aimed in the present invention could also be controlled by gene therapy in which the gene defect is corrected in situ or by the use of recombinant or other vehicles to deliver a DNA sequence capable of expressing the normal gene product whose effect counter balances the deleterious consequences of the disease mutation to the affected cells of the patient.

Toward the isolation and characterization of the gl gene, the inventors of the present invention have used a positional cloning approach. A detailed physical map was established using yeast and bacterial artificial chromosome (YACs, BACs). Transgenic mice were then generated with different BAC clones to localise the gl gene based on functional rescue of the gl osteopetrotic defect. The candidate glgene or region was isolated and sequenced. Finally, a large deletion in this candidate gene or region in gl mice that results in complete loss of gene expression was molecularly characterized.

Physical Mapping of the gl Gene

As an initial step in the positional cloning approach used by the inventors of the present application, the gI locus was localized genetically to the proximal portion of mouse chromosome 10 in a ˜1 cM interval. Vacher, J. and Bernard, H., Mammalian Genome, 10, 239-243,1999.

Interestingly, this study allowed the inventors to define cosegregation of the gl locus transmission with a congenic polymorphic region, potentially of 129Sv origin, maintained by brother-sister matings for more than one hundred generations. These polymorphic markers were used to screen five YAC libraries and allowed the applicant to establish a YAC contig covering ˜8.5 Mb.

To obtain genomic clones that would most probably be non-chimeric, a BAC contig was isolated and established. The BAC contig was composed of eighteen overlapping clones covering the gl candidate region. The markers D10 Mit184 and Cd24a were used as entry points and after several rounds of chromosome walking, a minimal candidate genomic interval of ∥500 kb was covered by the contig. Complete characterization of these clones and end insert probes from the BACs 545 M19 and 343 H5 delineated the non-recombinant interval, showing that the gl locus must lie between these two markers.

Functional Rescue in BAC Transgenic Mice

The strategy adopted by the inventor's of the present application was based on an in vivo biological activity test through functional rescue of the osteopetrotic gl/gl phenotype, using BAC transgenesis.

Three overlapping BACs (498 E23, 373 N3, 343 H5) covering ˜75% of the candidate region were injected. In contrast to non-transgenic grey homozygous gl/gl osteopetrotic littermates, all transgenic gl/gl animals carrying the BAC 373 N3 displayed normal growth, an agouti coat color and appropriate bone marrow development as demonstrated by histological analysis.

Transgene transmission followed Mendelian distribution, and complete rescue was observed in all gl/gl transgenic mice. No detrimental phenotype was noticed with age in transgenic animals.

These results suggested that the gl mutation was linked to a decreased activity of a gene included in the BAC 373 N3.

Identification and Characterisation of the ql Gene

To characterize the genes present on the BAC 373 N3, a shotgun M13 phage library was generated and sequenced. In parallel, BLAST searches against EST (Expressed Sequence Tags) databases and ORF (Open reading frame) prediction analyses were used to define transcription units and genes. Northern blot and RT-PCR gene expression analyses showed loss of expression of a unique ˜3 kb transcript in gl/gl animals.

Genomic structural c haracterization of the gl locus by Southern analysis defined six exons and five introns covering approximately 16780 base pairs for the wild-type gl locus. In contrast to the wild-type gl locus, genomic DNA from the gl/gl mice underwent a genomic rearrangement associated with a large ˜8 kb deletion, that included the gene promoter and a large part of the first exon.

This observation is consistent with the complete lack of detection of the gl messenger RNA.

Gl Polypeptide Structure and Localization

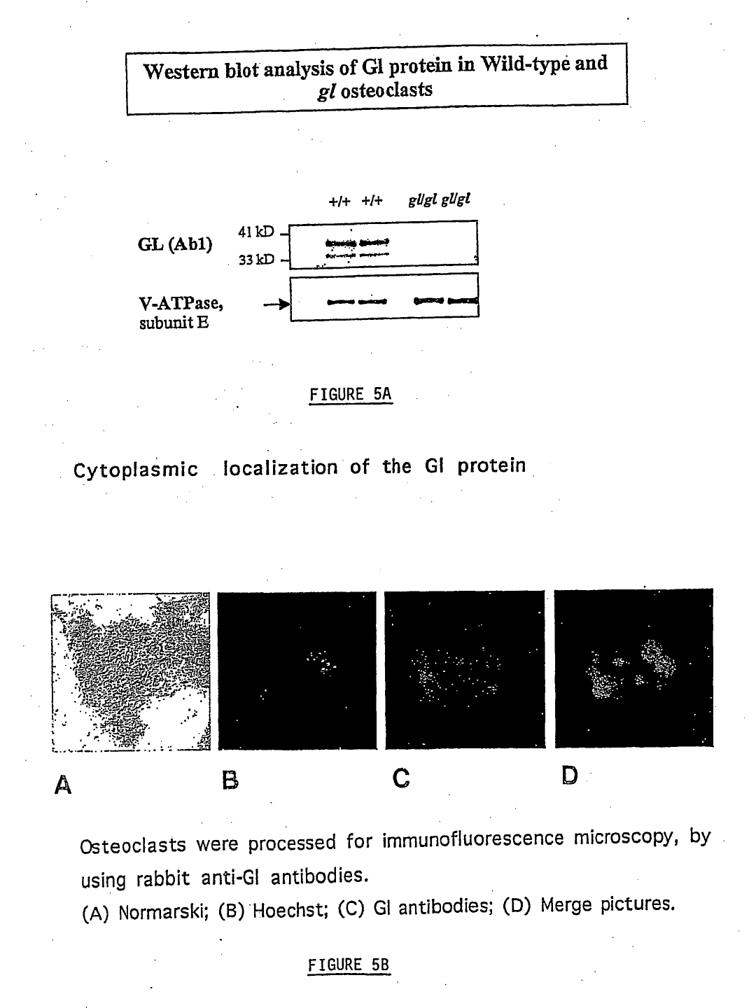

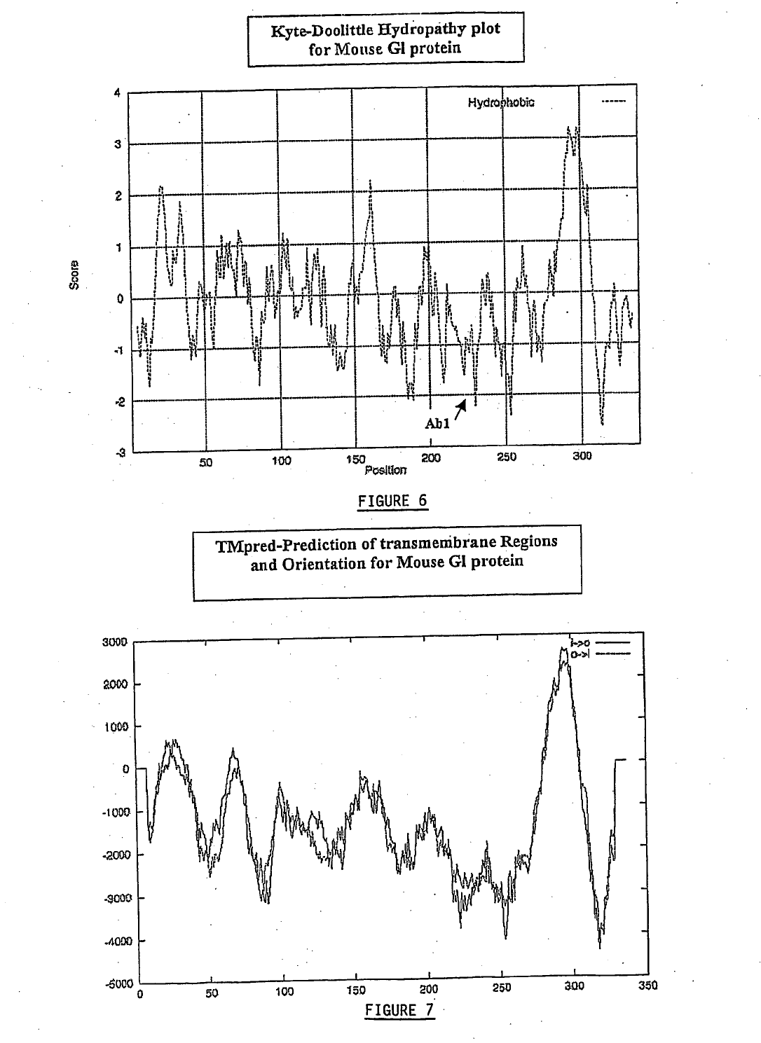

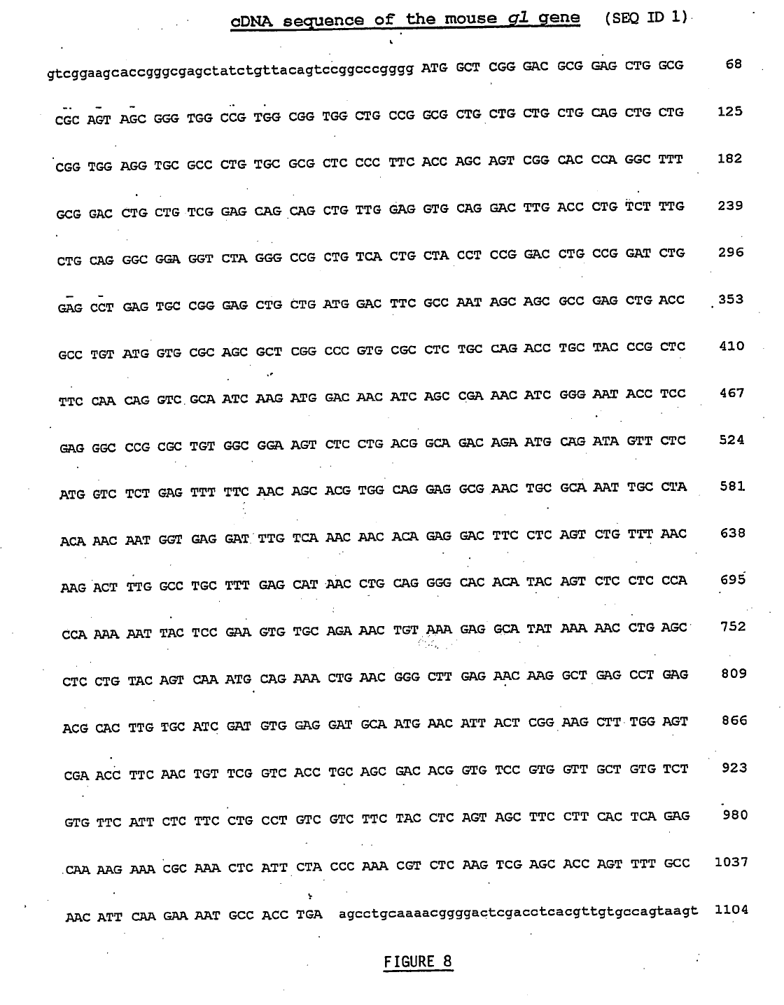

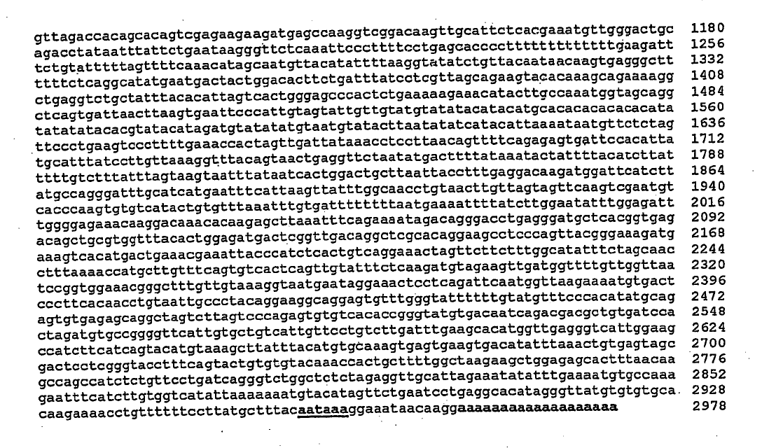

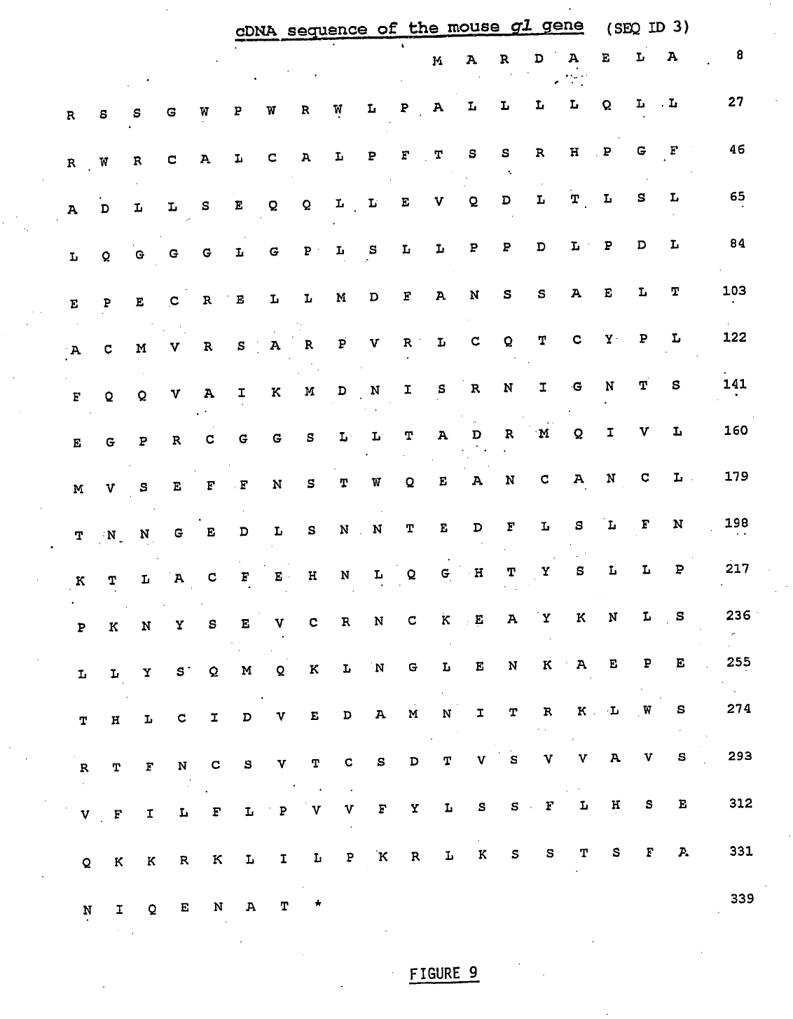

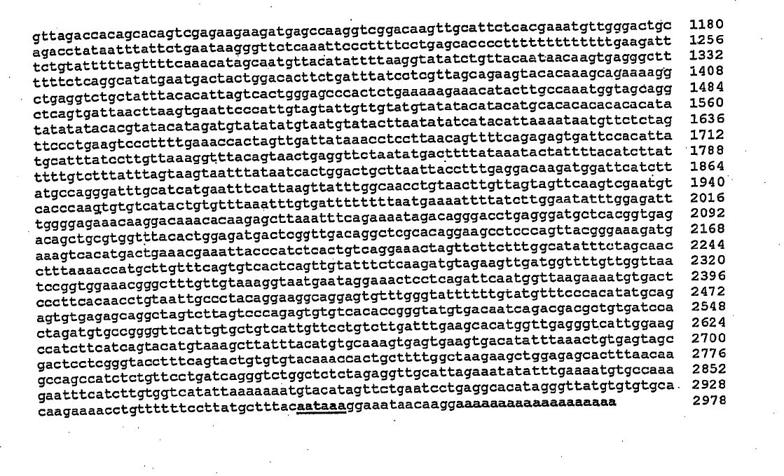

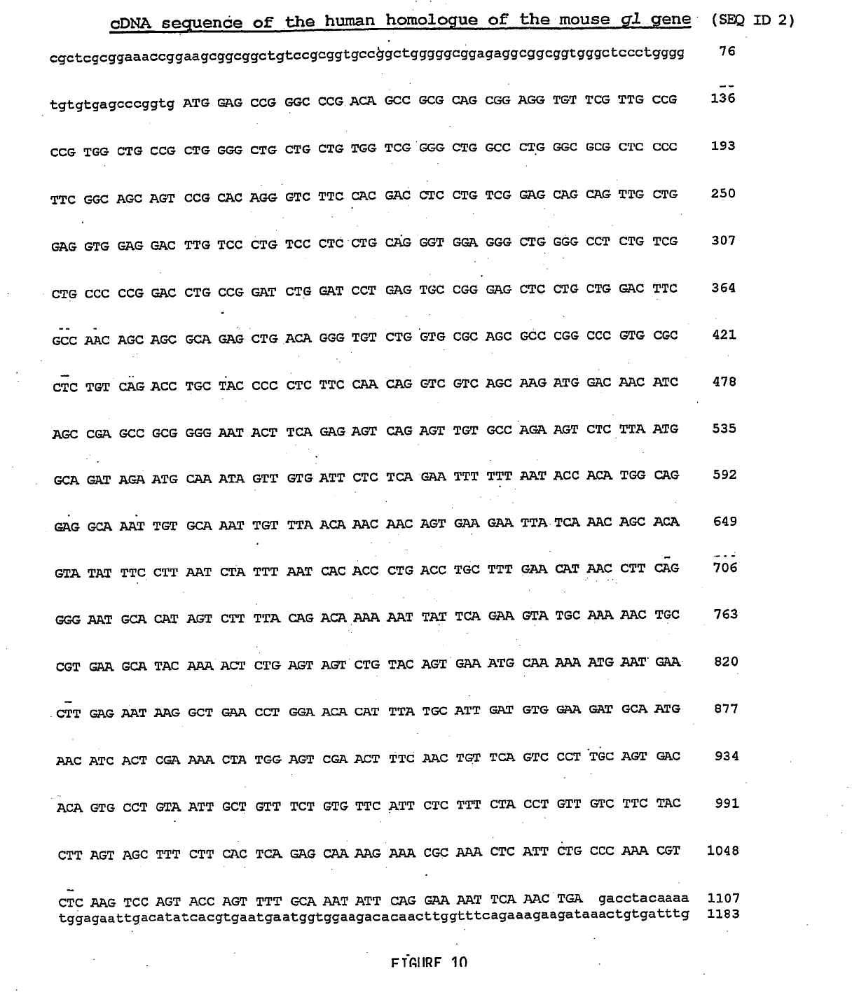

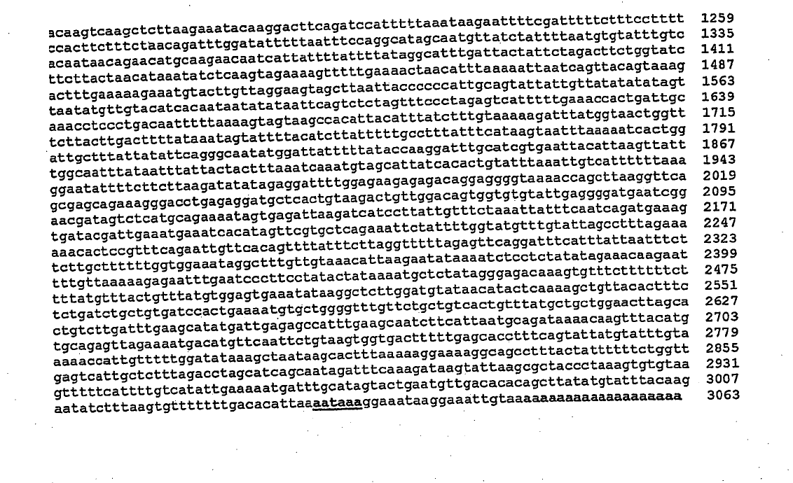

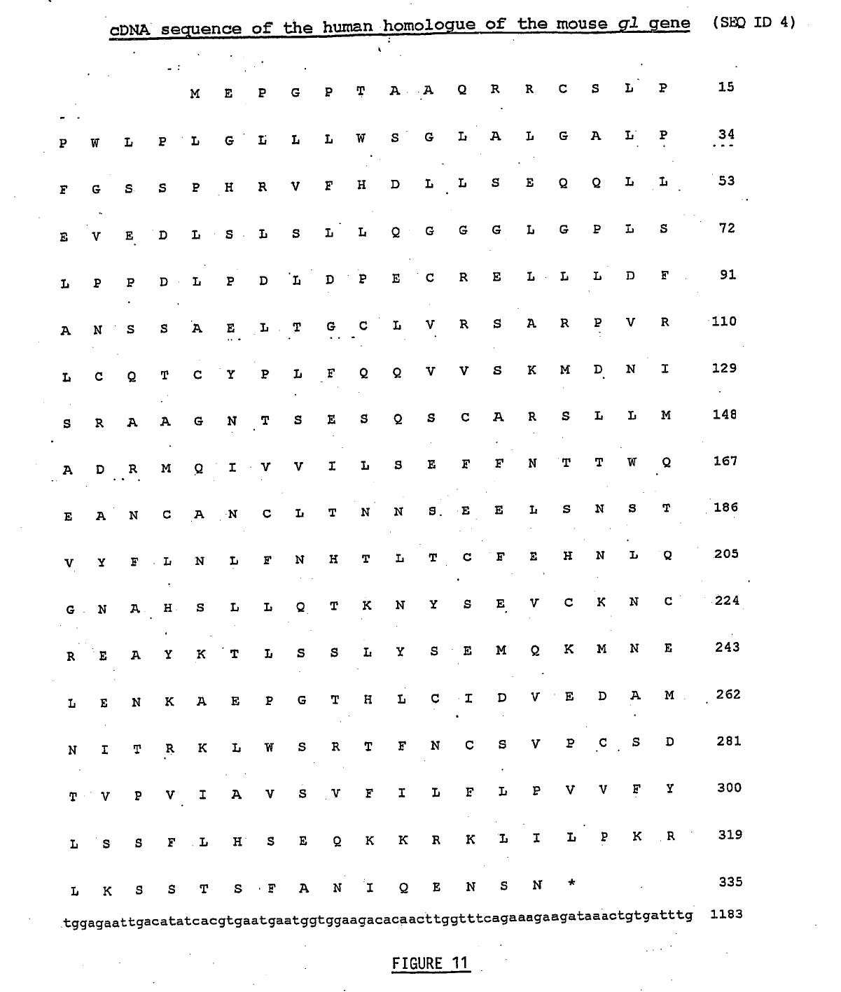

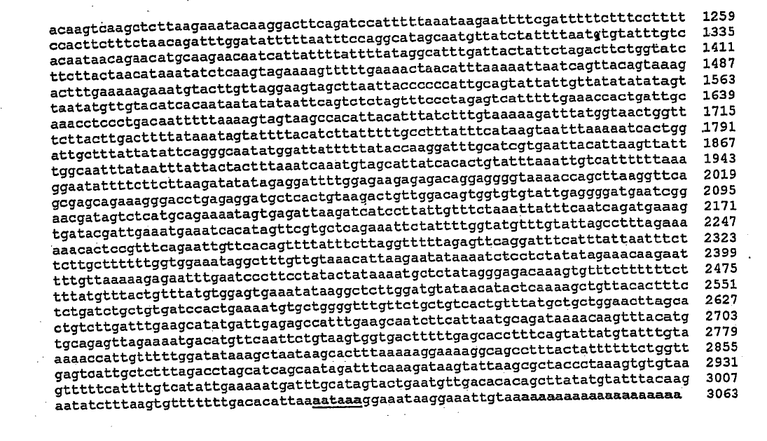

The open reading frame corresponding to the gl mRNA encodes a 338 amino acid protein with no obvious similarity with known protein sequences represented in protein databases. Hydropathy and protein topology analysis suggested the presence of one putative transmembrane domain in a protein enriched in cysteine residues. Two specific Gl antibodies corresponding to two different epitopes were used to detect by Western blot a ˜38 KDa protein in wild-type osteoclast extracts. In contrast no protein was detected in gl/gl cell extracts. Immunofluorescence analysis on wild-type native osteoclasts (FIG. 5), demonstrated specific cytosolic localization for the Gl polypeptide in multinucleated osteoclast as confirmed following Hoechst staining.

The analysis of predicted protein topology suggested that the protein has a putative transmembrane domain. Thus, this polypeptide may act as a receptor which has a binding specificity to a ligand which in turn has the function of modulating the activity of the Gl polypeptide; a channel protein; or a structural membrane protein.

Expression Pattern

Northern blot and RT-PCR analysis demonstrated a wide-spread expression pattern of a unique ˜3 Kb messenger RNA in several tissues including brain, spleen, liver, kidney, heart, thymus, testis and most importantly in osteoclast-like cells (OCLS) obtained in cocultures. Functional complementation was further correlated with detection of this specific transcript in rescued animals. Strong expression was detected in transgenic homozygous gl/gl tissues compared to the normal low level of expression in control non-transgenic wild-type littermate. This is in accordance with the high BAC transgene copy number (˜6) in this transgenic line. Furthermore, bone in situ hybridization demonstrated gl specific expression in multinucleated wild-type osteoclasts with higher expression in transgenic osteoclasts.

Gl polypeptide may be expressed using eukaryotic and prokaryotic expression systems. Eukaryotic expression system can be used for many studies of the gl gene and gene product including the determination of proper expression and post-translational modification for full biological activity, the identification of regulatory elements located in the 5 region of the production of large amounts of the normal and mutant protein for isolation and purification, to use cells expressing the Gl polypeptide as a functional assay system for antibodies generated against the protein and to test effectiveness of pharmacological agents or as a component of a signal transcription system to study the function of the normal complete protein, specific portion of the protein or of spontaneously occurring and genetically engineered mutant proteins.

One example of the prokaryotic expression system that may be used in the context of the present invention is the PET vector (Novagen).

Cloning of a Human Homoloques of the gl Gene

Database searches with the full length murine Gl polypeptide sequence identified homologous sequences in C. elegans and D. melanogaster. In contrast no human homologues were directly detected. However, highly conserved human EST clones were found and using genomic sequence of a PAC (Sanger center) combined with the mouse gene intron/exon structure, a gl human cDNA was assembled. The human sequence displayed high degree of conservation and close protein sequence identity with a 334 amino acids protein instead of 338 for the mouse protein.

The Gl polypeptide of the present invention comprises an amino sequence at least 89% identical to an amino acid sequence selected from the group consisting of SEQ ID NO:3, SEQ ID NO:4 and functional homologues thereof, exclusive of a NH2-terminal signal peptide (Target Program).

The Gl polypeptide of the present invention is also defined to comprise a nucleic acid sequence at least 90% identical to a sequence selected from the group consisting of SEQ ID NO:1, SEQ ID NO:2 and functional homologues thereof.

Antibodies for Detecting Gl Polypeptide

The present invention further provides an antibody that has a binding specificity to the Gl polypeptide of the present invention and fragments thereof.

Gl polypeptide antibodies can provide information on characteristic of the protein. For instance, generation of antibodies will enable the visualisation of the protein in cells and tissues using Western blotting.

In this technique, proteins are run on polyacrylamide gel and then transferred onto nitrocellulose membranes. These membranes are then incubated in the presence of the antibody (primary), then following washing are incubated with a secondary antibody which is used for detection of the protein-primary antibody complex. Following repeated washing, the entire complex is visualised using calorimetric or chemiluninescent assays.

Gl polypeptide antibodies also allow for the use of immunocytochemistry in immunofluorescent techniques in which the proteins can be visualised directly in cells and tissues. This is most helpful in order to establish the subcellular location of the protein and the tissues specificity of the protein.

In order to prepare polyclonal antibodies, fusion proteins containing defined portions or all of the Gl polypeptide can be synthesised in bacteria or in fungi by expression of corresponding DNA sequences in a suitable cloning vehicle. The protein can then be purified, coupled to a carrier protein and mixed with an adjuvant known by one skilled in the art suitable and injected into laboratory animals such as mice.

Alternatively, protein can be isolated from cultured cells expressing the protein. Following busters injections at bi-weekly intervals, the mice or other laboratory animals are then bled and the protein isolated. These sera can be used directly or purified pior to use, by various methods including affinity chromatography, protein A-sepharose, antigene sepharose, antimouse Ig-sepharose. The sera can then be used to probe protein extract run on a polyacrylimide gel to identify the Gl polypeptide. Alternatively, synthetic peptide can be made to the antigenic portion of the protein in use to inoculate the animals.

To produce monoclonal Gl polypeptide antibodies are prepared according to standard techniques known by one skilled in the art. For instance, cells actively expressing the protein are cultured or isolated from tissues and the cells membranes isolated. The membranes, extracts or recombinant protein extracts, containing the Gl polypeptide, are injected with an adjuvant into mice. After been injected nine times over a three weeks period, the mice spleens are removed and resuspended in phosphate saline buffer PSB. The spleen cells serve as a source of lymphocytes some of which are producing antibody of the appropriate specificity. These are then fused with a permanently growing myloma partner cell and the product of the fusion are plated under a number of tissue culture wells in the presence of selective agents, such as HAT. The wells are then screened to identify those containing cells making useful antibody by ELISA. These are then freshly plated. After a period of growth, these wells are again screened to identify antibody-producing cells. Several cloning procedure are then carried out until over 90% of the wells containing single clones, which are positive for antibody production. From this procedure to stable the line of clones is established which produce the antibody. The monoclonal antibody can then be purified by affinity chromatography using protein A sepharose, ion-exchange chromatography, as well as variation and combinations of these techniques.

In situ hybridisation is another method used to detect the expression of Gl polypeptide. In situ hybridisation relies upon the hybridisation of specifically labelled nucleic acid probe to the cellular RNA in individual cells or tissues. Therefore, it allows the identification of mRNA within intact tissues such as the brain. In this method, oligonucleotide corresponding to unique portions of the gl gene are used to detect specific mRNA species in the tissue of interest.

Antibodies may also be used coupled to compounds for diagnostic and/or therapeutic uses such as radionucleic for imaging and therapy and liposome for the delivering of compound to a specific tissue location.

Process for Producing the Gl Polypeptide

According to a preferred embodiment of the present invention, the Gl polypeptide is produced with a process comprising the step of culturing a host cell that is transformed or transfected with an expression vector comprising the nucleic acid or amino acid sequence of any one of SEQ ID NO:1 to 4, under condition suitable for the expression of the polypeptide.

In a preferred embodiment of the present invention, the host cell is a colony forming unit granulocyte macrophage selected from the group consisting of granulocyte macrophage lineage and monocyte.

In the alternative, the Gl polypeptide can be expressed in other cells such as insect cells using baculoviral vectors, or in mammalian cells using vaccinia virus or a specialised eukaryotic expression vectors. For expression in mammalian cells, the cDNA sequence may be ligated to heterologous promoters such as the simian virus (SV 40) promoter in the pSV2 vector or other similar vectors and introduced into cultured eukaryotic cells such as COS cells to achieve transit or a long term expression. The stable integration of the chimeric gene construct may be maintained in mammalian cells by biochemical selections such as neomycin and mycophoenolic acid.

Vectors are introduced into recipient cells by various methods including calcium phosphate, strontium, electroporation, lipofection, DEAE dextran, microinjection, or by photoplast fusion. Alternatively, the cDNA can be introduced by infection using viral vectors.

Using the techniques mentioned, the expression vectors containing the gl gene or portion thereof can be introduced into a variety of mammalian cells from other species or into non mammalian cells.

The recombinant cloning vector, according to this invention, comprises selected DNA of the DNA sequences of this invention for expression in a suitable host. The DNA is operatively linked in the vector to a promotor sequence in recombinant vehicle so that normal and/or mutant Gl polypeptide can be expressed. The expression controlled sequence will be selected from the group consisting of sequences that control the expression of genes of prokaryotic or eukaryotic cells and the viruses and combination therefore. The expression controlled sequence may be selected from the group consisting of the lac system, the trp system, the tac system, the trc system, major operator and promoter regions of phage lambda, the control region of the fd coat protein, promoter of SV 40, promoters derived from polyoma, adenovirus, baculovirus, 3-phophosglycerate kinase promoter, yeast promoters, combinations thereof.

The host cell which may be transfected with the vector of the present invention may be selected from the group consisting of bacteria, the yeast, fungi, insects, mouse or other animals, plant hosts or human tissue cells.

This process may further have a recovering and/or purifying step, wherein the polypeptide is recovered and/or recovered from the host cell through standard and well known procedures.

The Gl polypeptide may be isolated and purified by methods selected on the basis of properties revealed by its sequence. Since the protein processes properties of a membrane-spaning protein, a membrane fraction of cells in which the protein is highly expressed would be isolated and the proteins removed by extraction and the protein solubilised using a detergent.

Purification can be achieved using protein purification procedures, such as chromatography methods (gel, filtration, ion-exchange and immune affinity), by high performance liquid chromatography (RP-HPLC, ion exchange HPLC, size-exclusion HPLD and high performance chromatofocusing and hydrophobic interaction chromatography) or by precipitation (immuno precipitation). Polyacrylamide gel electrophoresis can also be used to isolate the Gl polypeptide based on its molecular weight, charge properties and hydrophobicity.

Similar procedures to those just mentioned could be used to purify the protein from cells transfected with vectors containing the Gl polypeptide (e.g. baculovirus systems, yeast expression systems, eukaryotic expression systems). Purified protein can be used in further biochemical analysis to establish secondary and tertiary structure, which may aid in the design of pharmaceuticals to interact with the protein or charge interaction with other proteins, lipid or saccharide moieties, alter its function in membranes as a transporter channel or receptor and/or in cells as an enzyme or structural protein in treated disease.

The protein may be in the form of a fusion protein Gl polypeptide-GST, which will facilitate its purification. For example, a fusion protein may be created by ligating the Gl cDNA sequence to a vector, which contains sequence for another peptide (e.g. GST-glutationine succinyl transferase). The fusion protein is expressed and recovered from a prokaryotic (e.g. bacterial or baculovirus) or an eukaryotic cell. The fusion protein can then be purified by affinity chromatography based upon the fusion vector sequence. The Gl polypeptide can then be further purified from the fusion protein by enzymatic cleavage of the fusion protein.

Therapies

Methods for preventing or treating a bone-related disease in a mammal are provided.

An important aspect of the biochemical studies using the genetic information of this invention is the development of therapies to circumvent or overcome the gl gene defects and thus prevent, treat, control serious symptoms or cure the disease. In view of expression of the gl gene in a variety of tissues, one has to recognise that other defects than osteoporosis and/or osteopetrosis may be caused by mutation in the gl gene in other tissues. Hence, in considering various therapies, it is understood that such therapies may be targeted at tissue other than the bone marrow, such as the heart, the testis, the spleen and the kidneys, where Gl polypeptide is also expressed.

In a particular embodiment, the method comprises modulating the expression of the nucleic acid and/or the concentration of the Gl polypeptide of the present invention. The expression and/or concentration of the osteoclast-related polypeptide may be increased thereby preventing or treating a lack of bone resorption, such as osteopetrosis, in the mammal subject.

In this embodiment, the expression of the nucleic acid or the concentration of the polypeptide is increased by administering to the mammal subject at least one of the following: a functional nucleic acid molecule of the present invention; an expression or cloning. vector having the nucleic acid molecule of the present invention; a host cell comprising the latter; a molecule for activating in said mammal the expression of the above mentioned nucleic acid molecule; an Gl polypeptide of the present invention; a molecule for activating the production or increasing the concentration of the Gl polypeptide of the invention; and a viral vector having the nucleic acid sequence of the invention.

In another embodiment, the expression and/or concentration of the Gl polypeptide of the present invention is reduced, thereby treating or preventing an excess of bone resorption, such as osteoporosis in a mammal subject.

In this embodiment, the expression and/or concentration of the Gl polypeptide is reduced by administering to the mammal subject at least one of the following molecule: a molecule having the function of inhibiting the expression of the nucleic acid sequence encoding for the Gl polypeptide of the invention; a molecule for inhibiting the production of the Gl polypeptide of the invention; and/or a molecule having the function of reducing the concentration of the Gl polypeptide of the invention.

The molecule which has the function of inhibiting the expression of the nucleic acid mentioned herein above may be one that, for instance, binds to the nucleic acid thereby blocking the transcription and/or the translation steps of the polypeptide thus inhibiting its production.

In the case where the Gl polypeptide of the present invention is a receptor or an ion channel protein, a test for osteoporosis or osteopetrosis can be produced to detect an abnormal receptor or an abnormal function related to abnormalities that are inquired or inherited in the gl gene and its product, or in one of the homologues genes and their products. This test can be accomplished either in vivo or in vitro by measurements of ion channel fluxes and/or transmembering voltage or current fluxes using patch, clamp, voltage clamp and fluorescent dies since it is to intracellular calcium or transmembrane voltage. Defective ion channel or receptor function can also be assayed by measurements of activation of second messengers such as cyclic AMP, cGMP kinases, phosphates, increases in intracellular Ca2⁺ levels, etc. Recombinantly made protein may also be reconstructed in artificial membrane systems to study ion channel conductance.

Therapies which affect bone related diseases can be tested by analysis of their ability to modify an abnormal ion channel or receptor function mutation in the gl gene in one of its homologues. Therapies could also be tested by their ability to modify the normal function of an ion channel or receptor capacity of the gl gene products and its homologues. Such assays can be performed on cultured cells expressing endogenous normal or mutant gl genes/gene products (or its homologues). Such studies can be performed in additional cells transfected with vectors capable of expressing Gl polypeptide, parts of the gl gene and gene product, mutant Gl polypeptide or of its homologues (abnormal or mutant form).

Therapies for bone related diseases could be divided to modify an abnormal ion channel or receptor function of the gl gene or its homologues. Such therapies can be conventional drugs, peptides, sugars or lipids as well as antibodies or other agents, which affect the properties of the gl gene product. Such therapies can also be performed by direct replacement of the gl gene and/or its homologues by gene therapy. in the case of an ion channel, the gene therapy could be performed using either many genes (cDNA+a promoter) or a genomic construct bearing genomic DNA sequences for parts or all of the gl gene. Mutant Gl polypeptide or homologous gene sequences might also be used to counter the effect of the inherited or acquired abnormalities of the gl gene. The therapy may also be directed at augmenting the receptor Gl channel function of the homologues genes in order that it may potentially take over the functions of the gl gene rather defective by acquired or inherited defects. Therapies using antisence oligonucleotides to block the expression of the mutant gl gene coordinated with gene replacement with normal Gl polypeptide or a homologue gene can also be applied using standard techniques or either gene therapy or protein replacement therapy.

Pharmaceutical Preparation

A pharmaceutical composition for preventing or treating osteopetrosis is provided. The composition comprises in a pharmaceutically effective amount a molecule that has the function of increasing the expression and/or concentration of the Gl polypeptide of the present invention. This molecule may be selected from the group of molecule used to prevent or treat osteopetrosis mentioned in the previous section.

A pharmaceutical composition for preventing or treating osteoporosis is also provided. The composition comprises in a pharmaceutically effective amount of a molecule having the function of reducing the expression and/or concentration of the Gl polypeptide of the present invention. This molecule may be selected from the group of molecule used for treating or preventing osteoporosis mentioned in the previous section.

The term “pharmaceutically effective amount” means an amount, which provides a therapeutic effect for a specified condition and route of administration.

According to various embodiments of the present invention, the pharmaceutical composition may further comprise pharmaceutically acceptable diluant, carrier, solubiliser, emulsifier, preservative and/or adjuvant.

The composition may be in a liquid or lyophilised form and comprises a diluant (Tris, acetate or phosphate buffers) having various pH values and/or ion exchange; solubiliser such as Tween or polysorbate; carriers such as human serum, albumin or gelatine; preservatives such as thimerosal or benzyl alcohol and antioxidants such as ascorbic acid or sodium metabisulfite.

The composition of the invention may be in solid or liquid form or any suitable form for a therapeutic use. They may be formulated for a rapid or slow release of its components. The composition of the invention may be prepared according to conventional methods known in the art.

Kit for Screening the Gl Polypeptide Molecule of the Present Invention

Screening for a human related disease such as osteopetrosis and/or osteoporosis as link to chromosomes 6 may now be really carried out because of the knowledge of the location of the gene.

People with high risk for osteopetrosis or osteoporosis (person in family pedigree) or individuals not previously known to be at high risk, or people in general may be screened routinely using probes to detect the presence of a mutant gl gene by a variety of techniques.

Genomic DNA used for the diagnosis may be obtained from body cells, such as those present in the blood, tissue biopsy, and surgical specimens or autopsy material. The DNA may be isolated and used directly for detection of its specific sequence or may be amplified part to analysis. RNA or cDNA may also be used.

To detect a specific DNA sequence, hybridisation using a specific oligonucleotide, direct DNA sequencing, restriction enzyme digest, RNase protection, chemical cleavage and ligase-mediated detection are all methods, which can be utilised.

Oligonucleotides specific to mutant sequences can be chemically synthesised and labelled radioactively with isotopes or non-radioactive using biotin tags and hybridised to individual DNA samples immobilised on membranes or other solid supports by dot-blot or transfer from gels after electrophoresis. The presence or absence of these mutant sequences is then visualised using methods such as autoradiography, fluorometry or colormetric reaction.

Direct DNA sequencing reveals sequence differences between normal and mutant OR polypeptide DNA. Cloned DNA segments may be used as probes to detect specific DNA segments. PCR can be used to enhance the sensitivity of this method by exponentially increasing of the target DNA.

Other nucleotide sequence simplification techniques may be used such as ligation-mediated PCR, anchored PCR and enzymatic amplification as would be understood by those skilled in the art.

Sequence alteration may also generate fortuitous restriction enzyme recognition sites, which are revealed by the use of appropriate enzyme digestion followed by gel electrophoresis and blot hybridisation. DNA fragments carrying the site (normal or mutant) are detected by their increased reduction size or by the increase of corresponding restriction family numbers. Genomic DNA samples may also be amplified by PCR prior to treatment with appropriate restriction enzyme and the fragments of different sizes are visualised under UV light in the presence of ethidium bromide after gel electrophoresis.

Genetic test is based on DNA sequence differences may be achieved by detection of alteration in electrophoretic mobility of DNA fragments in gels. Small sequence deletion and insertion can be visualised by high resolution gel electrophoresis. Small deletions may also be detected as changes in the migration pattern of DNA heteroduplexes in non denaturing gel electrophoresis. Alternatively a single base substitution or deletion mutation may be detected based on differential PCR product length in PCR. The PCR products for the normal and mutant gene could be differentially detected in acrylamide gels.

A kit for screening a nucleic acid sequence encoding for the Gl polypeptide of the present invention is provided. The kit comprises a nucleic acid probe or primer complementary to the nucleic acid sequence of the present invention; reagents for hybridization of the probe or primer to a complementary nucleic acid sequence; and means for detecting hybridization.

The present invention also provides a kit for detecting the presence of the Gl polypeptide of the present invention.

In this embodiment, the kit comprises a probe or primer having a binding specificity to the Gl polypeptide of the present invention; reagents for hybridization of the probe or primer to the Gl polypeptide; and means for detecting hybridization.

### EXAMPLE 1

Mice

The mouse strain GL/Le dl+/+gl was purchased from the Jackson Laboratory (Bar Harbor, Me.). Homozygous gl/gl mice were generated by breeding heterozygous gl/+animals, and displayed a typical grey coat color instead of agouti, a major growth retardation and a lack of tooth eruption. All animals produced from these matings were genotyped at the gl locus by using cosegregating polymorphic markers that we have previously described. Vacher, J and Bernard, H., Mamm. Genome,10, 239-243,1999.

### EXAMPLE 2

BAC Library Screening and Contig Establishment

The 129/Sv CITB mouse BAC library (Research Genetics, Huntsville, Ala.) was screened by PCR using the markers D10Mit184. Amplification reaction was performed in a 20 μl of 10 mM Tris-hydroxychloride, pH 8.3, 50 mM KCl and 1.5 mM MgCl2. Each reaction contained 10 ng of DNA, 0.5 μM of each primer, 0,2 mM dNTP and 1U Taq polymerase (GIBCO-BRL). Thermal cycler conditions were 94° C., 5 min and 30 cycles (94° C., 1 min; 55° C., 1 min; 72° C., 2 min). PCR reactions were analyzed by gel electrophoresis on 10% acrylamide slab gel and specific products detected by ethidium staining.

A last round of screening on filter was carried out using BAC insert ends as probes. In brief, the membrane was prehybridized 2 h at 65° C. in 5×SSC 5× Denhardt's solution, 0.5% SDS and 10 mg/ml of sonicated denatured salmon sperm DNA, followed by hybridization with the BAC end probes overnight at 65° C. in the same solution. The membrane was washed twice at 65° C. in 1×SSC/0.1% SDS for 20 min and exposed to X-ray film. The size of each clone was determined by pulsed-field gel electrophoresis. By this approach, we have established a contig of 18 adjacent clones using overlapping PCR assays derived from BAC end sequences and polymorphic markers.

### EXAMPLE 3

Library Screening and cDNA Isolation

To isolate the full-length gl cDNA, we screened a C57BL/6 spleen cDNA mouse library (Stratagene) by PCR. Takumi, T. and Lodish, H. F. BioTechniques, 17:443-444 (1994). This library was divided in 16 pools, each of which contained approximately 100,000 clones. PCR assays were conducted with gl forward 5′-GGCGAGCTATCTGTTACAGTCC-3′ and gl reverse 5′-TTACTGGCACAACGTGAGGTC-3′ primers. PCR amplification conditions were 94° C., 5 min and 30 cycles (94° C., 1 min; 63° C., 1 min; 72° C., 2 min) in 20 mM Tris-HCl, pH 8.4, 50 mM KCl, 2 mM MgCl2, 5% DMSO with 0.5 mM dNTPs, 0.5 μM primers and 1U Taq polymerase in 20 μl volume. The last step of screening consisted of filter hybridization in the same conditions as described above. The cDNA was then sequenced (Thermosequenase, Amersham) and the protein open reading frame deduced.

### EXAMPLE4

Expression Analysis

Expression analysis of the gl gene was carried out by both Northern blots and RT-PCR analysis.

First we have isolated total RNAs from adult mouse whole brain, liver, spleen, kidney, heart, thymus and testis tissues by a standard LiCl/Urea method as previously described. Vacher, J. and Tilghman, S. M. Science, 250:1732-1735 (1990). Total RNAs from osteoclast-like cells (OCLs) were isolated by TRIzol (Gibco BRL) as previously described. Rajapurohitam, V. et al. Bone, 28:513-523 (2001).

For Northern analysis 15 μg of total RNA or 2 μg of polyA⁺ RNA were fractionated by 1.5% agarose/2.2M formaldehyde gel electrophoresis and transferred onto membrane. The membrane was prehybridized 2 h at 65° C. in 5×SSC, 5× Denhardt's solution, 0.5% SDS and 10 mg/ml sonicated denatured salmon sperm DNA, and hybridized overnight at 65° C. in the same solution with a radiolabelled 1.9 kb gl cDNA probe. The membrane was washed twice at 65° C. in 2×SSC/0.1% SDS for 20 min. and exposed to X-ray film.

For RT-PCR analysis, reverse transcription (Superscript II, Gibco BRL) of 1 μg of RNA with oligo dT primer was conducted in 20 mM Tris-HCl, pH 8.4, 50 mM KCl, 1.5 mM MgCl2 with 0.5 mM dNTPs, 0.5 mM primers and 1U Taq polymerase in a 20 μl volume. PCR amplification conditions of 1 μl of cDNA were 94° C., 5 min and 30 cycles (94° C., 1 min; 60° C., 1 min; 72° C., 2 min). The gl primers were: Forward 5′-CCTGCTTTGAGCATAACCTGC-3′ and Reverse 5′-TTACTGGCACAACGTGAGGTC-3′ and for beta-actin control were Forward 5′-TGACGATATCGCTGCGCTG-3′ and Reverse 5′-ACATGGCTGGGGTGTTGAAG-3′. PCR products were analyzed on 1% agarose gels and detected by ethidium bromide staining. Generation of BAC transgenic mice and histologic analysis

### EXAMPLE 5

Generation of BAC Transgenic Mice and Histologic Analysis

Circular BAC DNA (1 ng/μl) was injected into fertilized mouse oocytes isolated from F1 (C3H×C57BL/6)×C57BL/6 crosses. Transgenic founders were identified by PCR using specific BAC end sequence assay and internal polymorphic markers. Each founder was first crossed with heterozygote gll+mice, and gll+transgenic progeny were intercrossed. The gl/gl transgenic mice were then identified by homozygosity at the polymorphic D10Mit184, D10Mit108 and D10Mit255 loci. Vacher, J and Bernard, H., Mammalian. Genome, 10, 239-243,1999.

Histology was done on bone samples fixed in 10% phosphate-buffered formalin, decalcified in 14% EDTA, and embedded in paraffin. Adjacent sections (6 μm) were stained with hematoxylin and eosin.

### EXAMPLE 6

Gl Gene Structure

Intron-exon boundaries were characterized following alignement of the complete mouse cDNA sequence against mouse genomic sequences obtained by BLAST searches from NCBI Genomic Survey Sequence (GSS) and NCBI mouse Trace archive. Each intron-exon junction corresponds to the loss of alignement between cDNA and genomic sequences (usually at splicing consensus sites GT/AG).

Introns size was estimated by restriction mapping of genomic DNA, followed by membrane transfer and Southern blot hybridization (conditions described above) with various parts of the gl cDNA as probes.

Genomic DNA was prepared from tail biopsies as previously described. Laird, P W et al. Nucl Ac Res 19:4293 (1994). After restriction digests (BamHI, BgIII, EcoRI), Southern blots were hybridized with gl cDNA probes in the same conditions as described above. The membrane was washed twice at 65° C. in 1×SSC/0.1% SDS for 20 min. and exposed to X-ray film.

### EXAMPLE 7

Protein Extracts, GL Antibodies and Western Blotting

OCLs were obtained by co-culturing one-day-old FVB/NJ calvarial osteoblasts and spleen cells of either +/+ or gl/gl mouse as previously described. Rajapurohitam, V. et al., Bone, 28:513-523 (2001). Cultured OCLs were washed twice with phosphate buffered saline and lysates were prepared in ice cold cell lysis buffer (50 mM sodium pyrophosphate, 50 mM sodium fluoride, 50 mM NaCl, 5 mM EDTA, 5 mM EGTA, 2 mM sodium ortho vanadate, 10 mM HEPES, 0.1% Triton X-100, 0.05% NP-40) in the presence of protease inhibitors. Lysates were sonicated for 30 sec, incubated on ice for 30 min and centrifuged at 12,000×g for 20 min at 4° C. Supernatants were collected and protein concentrations determined by Bradford assay using BSA as the standard.

Rabbit polyclonal antibodies Ab1 and Ab2 were raised against multiple-antigen peptides MAP1: LHSEQKKRKLILPKR-MAP and MAP2 LNGLENKAEPETHLC-MAP respectively. Protein extracts (25 μg) were resolved on 12% SDS-PAGE gels and transferred onto nitrocellulose membranes. Following transfer, membranes were stained with Ponceau red to confirm uniform transfer and proteins integrity. Membranes were incubated in 5% milk for 1 h and then washed twice 10 min in Tris buffered saline Tween (TBS-T). Membranes were probed with polyclonal antisera (Ab1, 1:100 dilution; Ab2, 1:100 dilution or 31 kDa V-ATPase subunit, 1:500 dilution in TBS-T, 3% BSA) for 1 h at room temperature. Membranes were washed twice 10 min in TBS-T, incubated with horseradish peroxidase-protein-A (HRP-A) secondary antibodies for 1 h at room temperature. After TBS-T washing, the signal was revealed by the ECL western blotting detection reagent (Amersham) and exposed on film.

### EXAMPLE 8

In Situ Hybridization and Immunofluorescence

In situ hybridization was done as described previously. Emerson et al. Dev. Dynamics 195: 55-66, 1992. Bone samples were fixed in 10% phosphate-buffered formalin, decalcified in 14% EDTA, and embedded in paraffin. Paraffin was removed in xylenes and sections were fixed in 4% paraformaldehyde and hybridized to α-S³⁵UTP-labeled riboprobes overnight at 55° C. The gl antisense riboprobe was generated by T7 polymerase transcription of the 0.5 kb 3′UTR fragment cloned into Bluescript and linearized by Spel. The sense riboprobe was generated by T3 transcription of the same template linearized by Kpnl. Hybridized sections were dipped in K2 photographic emulsion, exposed 2-3 weeks at 4° C., developped using D-19 developer and general fixer (Kodak) and stained with hematoxylin and eosin.

Immunofluorescence was conducted on isolated wild-type osteoclasts isolated from three-days old pups and cultured overnight on slides in α-MEM with 10% fetal calf serum, in 5% CO2. Slides were washed in phosphate-buffered saline (PBS) and the cells were fixed in 4% paraformaldehyde in PBS for 10 min. Samples were then incubated at room temperature for 1 hr in PBS containing 0.1% BSA, 0.05% saponin and 5% normal goat serum to block non-specific binding, and subsequently for 1 hr with Gl primary antibody (1:50). Slides were then washed in PBS and incubated with secondary AlexaFluor 488-conjugated goat anti-rabbit IgG antibodies (1:100; Molecular Probes) for lhr in the dark. For Hoechst staining, slides were incubated in 1:1500 dilution in water of a 0.5 mg/ml Hoechst 33258 at room temperature for 10 min. After washing with PBS, samples were mounted in FluorSave (Calbiochem) and cells were visualizecl by confocal laser scanning microscopy (Axiophot, Zeiss).

Genomic structure of the mouse gl gene

| Exon | Exon | CDNA^{a} | Exon/Intron junction sequences^{c} | | Intron | Intron^{b} |
|---|---|---|---|---|---|---|
| No. | Length (bp) | Position | Splice donor | splice accepo | No. | Length (Kbp) |
| 1 | 458 | 1-458 | ATCGGGgtgggt | TtgcagAATACC | 1 | ˜3.9 |
| 2 | 115 | 459-573 | GCGCAAgtgagt | TtacagATTGCC | 2 | ˜3.6 |
| 3 | 98 | 574-671 | CTGCAGgtcagt | TtttagGGGCAC | 3 | ˜2.8 |
| 4 | 168 | 672-839 | GATGCAgtgagt | TtctagATGAAC | 4 | ˜1.7 |
| 5 | 166 | 840- | TTCTACgtaagt | CcccagCCAAA | 5 | ˜1.8 |
| 6 | 1973 | 1006-2978 | 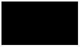 | 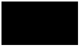 | 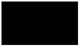 | 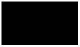 |

^{a}: cDNA sequence was obtained from clones isolated by screening the STRATAGENE and CLONETECH spleen libraries and comparing their sequences to the corresponding ESTs from GenBank and Riken database.
^{b}: nintron size was estimated from restriction mapping analysis.
^{c}: Exon sequences are in uppercase letters, intron sequences are in lowercase.

Genomic structure of the human homologue of the mouse gl gene

| Exon | Exon | cDNA^{a} | Exon/Intron junction sequences^{c} | | Intron | Intron^{b} |
|---|---|---|---|---|---|---|
| No. | Length (bp) | Position | Splice donor | splice accepto | No. | Length (Kbp) |
| 1 | 493 | 1-493 | GCGGGGgtggg | TtacagAATACT | 1 | 9.95 |
| 2 | 115 | 494-608 | GTGCAAgtaagt | TgacagATTGTT | 2 | 9.597 |
| 3 | 98 | 609-706 | CTTCAGgtattt | TtttagGGGAAT | 3 | 3.291 |
| 4 | 168 | 707-874 | GATGCAgtaagt | TtccagATGAAC | 4 | 1.612 |
| 5 | 166 | 875-1040 | TTCTGCgtaagt | AtctagCCAAAC | 5 | 4.412 |
| 6 | 2023 | 1041-3069 | 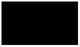 | 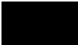 | 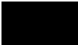 | 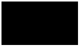 |

^{a}: cDNA sequence was obtained by aligning the mouse cDNA sequence against the Genbank ESTs database.
^{b}: Genomic sequence was obtained from the sequenced human PAC (RP1-111B22) at Sanger center (Acc No.: 798200).
^{c}: Exon sequences are in uppercase letters, intron sequences are in lowercase.

1-46. (canceled)

47. A method for preventing or treating a bone resorption-related disease in a mammal subject, comprising modulating in said mammal subject:
(a) the expression of a nucleic acid sequence at least 90% identical to a sequence selected from the group consisting of SEQ ID NO: 1 and SEQ ID NO: 2, and/or
(b) the concentration of an GI polypeptide comprising an amino acid sequence that is encoded by said nucleic acid sequence.

48. The method according to claim 47, wherein said nucleic acid sequence is 100% identical to a sequence selected from the group consisting of SEQ ID NO: 1, SEQ ID NO: 2, and fragments thereof.

49. The method according to claim 48, wherein said nucleic acid sequence is SEQ ID NO: 2.

50. The method according to claim 47 wherein said amino acid sequence is at least 80% identical to an amino acid sequence selected from the group consisting of SEQ ID NO: 3 and SEQ ID NO: 4.

51. The method according to claim 50, wherein said amino acid sequence is 100% identical to an amino acid sequence selected from the group consisting of SEQ ID NO: 3, SEQ ID NO: 4, and fragments thereof.

52. The method according to claim 51, wherein said amino acid sequence is SEQ ID NO: 4.

53. The method according to claim 47, wherein said GI polypeptide comprises a transmembrane domain.

54. The method according to claim 53, wherein said GI polypeptide is a receptor.

55. The method according to claim 54, wherein said expression of said nucleic acid sequence and/or said concentration of said GI polypeptide is increased, thereby preventing or treating a lack of bone resorption in said mammal subject.

56. The method according to claim 55, wherein said expression of said nucleic acid sequence and/or said concentration of said GI polypeptide is increased by administering to said mammal subject at least one of the following:
(a) a functional nucleic acid sequence at least 90% identical to a sequence selected from the group consisting of SEQ ID NO: 1 and SEQ ID NO: 2,
(b) an expression or cloning vector having said nucleic acid sequence,
(c) a molecule for activating, in said mammal subject, said expression of said nucleic acid sequence,
(d) said GI polypeptide,
(e) a molecule for activating, in said mammal subject, the production of said GI polypeptide,
(f) a molecule for increasing, in said concentration of said GI polypeptide,
(g) a host cell transformed or transfected with said expression or cloning vector, and
(h) a viral vector having said nucleic acid sequence.

57. The method according to claim 47, wherein said bone resorption-related disease is osteopetrosis.

58. The method according to claim 47, wherein said expression of said nucleic acid sequence and/or said concentration of said GI polypeptide is reduced, thereby preventing or treating an excess of bone resorption in said mammal subject.

59. The method according to claim 58, wherein said expression of said nucleic acid sequence and/or said concentration of said GI polypeptide is reduced by administering to said mammal subject at least one of the following:
(a) a molecule having the function of inhibiting said expression of said nucleic acid sequence.
(b) a molecule having the function of inhibiting said production of said GI polypeptide, and/or
(c) a molecule having the function of reducing said concentration of said GI polypeptide.

60. The method according to claim 59, wherein said molecule having the function of inhibiting said expression of said nucleic acid sequence is a molecule that binds therewith.

61. The method according to claim 59, wherein said molecule having the function of inhibiting said production of said GI polypeptide is a molecule that blocks the translation of said Gl polypeptide.

62. The method according to claim 57, wherein said bone resorption-related disease is osteoporosis.

63. A method of making a pharmaceutical composition for treating or preventing excess bone resorption in a mammal comprising combining at least one of the following:
(a) a functional nucleic acid sequence at least 90% identical to a sequence selected from the group consisting of SEQ ID NO: 1 and SEQ ID NO: 2,
(b) an expression or cloning vector having said nucleic acid sequence,
(c) a molecule for activating the expression of said nucleic sequence,
(d) an GI polypeptide comprising an amino acid sequence that is encoded by said nucleic acid sequence,
(e) a molecule for activating the production of said GI polypeptide,
(f) a molecule for Increasing the concentration of said GI polypeptide,
(g) a host cell transformed or transfected with said expression or cloning vector, and
(h) a viral vector having said nucleic acid sequence, with a pharmaceutically acceptable excipient.

64. The method according to claim 63, wherein said lack of bone resorption is osteopetrosis.

65. A method of making a pharmaceutical composition for treating or preventing excess bone resorption in a mammal comprising combining at least one of the following:
(a) a molecule having the function of inhibiting the expression of a nucleic acid sequence at least 90% identical to a sequence selected from the group consisting of SEQ ID NO: 1 and SEQ ID NO: 2,
(b) a molecule having the function of inhibiting the production of an GI polypeptide comprising an amino acid sequence that is encoded by said nucleic acid sequence, and/or
(c) a molecule having the function of reducing the concentration of said GI polypeptide, with a pharmaceutically acceptable excipient.

66. The method according to claim 65, wherein said excess of bone resorption is osteoporosis.

67. The method according to any one of claims 63 to 65, wherein said nucleic acid sequence is 100% Identical to a sequence selected form the group consisting of SEQ ID NO: 1, SEQ ID NO: 2, and fragments thereof.

68. The method according to claim 67, wherein said nucleic acid sequence is SEQ ID NO: 2.

69. The method according to claim 68, wherein said amino acid sequence is at least 80% identical to an amino acid sequence selected from the group consisting of SEQ ID NO: 3 and SEQ ID NO: 4.

70. The method according to claim 69, wherein said amino acid sequence is 100% identical to an amino acid sequence selected from the group consisting of SEQ ID NO: 3, SEQ ID NO: 4, and fragments thereof

71. The method according to claim 70, wherein said amino acid sequence is SEQ ID NO: 4.

72. The method according to claim 71, wherein said GI polypeptide comprises a transmembrane domain.

73. The method according to claim 72, wherein said GI polypeptide is a receptor.


SEQUENCE LISTING (31 sequences)

SEQ ID NO: 1 - DNA, 2997 bp, Mus musculus
gtcggaagcaccgggcgagctatctgttacagtccggcccggggatggctcgggacgcggagctggcgcgcagtagcgggtggccgtggcggtggctgccggcgctgctgctgctgcagctgctgcggtggaggtgcgccctgtgcgcgctccccttcaccagcagtcggcacccaggctttgcggacctgctgtcggagcagcagctgttggaggtgcaggacttgaccctgtctttgctgcagggcggaggtctagggccgctgtcactgctacctccggacctgccggatctggagcctgagtgccgggagctgctgatggacttcgccaatagcagcgccgagctgaccgcctgtatggtgcgcagcgctcggcccgtgcgcctctgccagacctgctacccgctcttccaacaggtcgcaatcaagatggacaacatcagccgaaacatcgggaatacctccgagggcccgcgctgtggcggaagtctcctgacggcagacagaatgcagatagttctcatggtctctgagtttttcaacagcacgtggcaggaggcgaactgcgcaaattgcctaacaaacaatggtgaggatttgtcaaacaacacagaggacttcctcagtctgtttaacaagactttggcctgctttgagcataacctgcaggggcacacatacagtctcctcccaccaaaaaattactccgaagtgtgcagaaactgtaaagaggcatataaaaacctgagcctcctgtacagtcaaatgcagaaactgaacgggcttgagaacaaggctgagcctgagacgcacttgtgcatcgatgtggaggatgcaatgaacattactcggaagctttggagtcgaaccttcaactgttcggtcacctgcagcgacacggtgtccgtggttgctgtgtctgtgttcattctcttcctgcctgtcgtcttctacctcagtagcttccttcactcagagcaaaagaaacgcaaactcattctacccaaacgtctcaagtcgagcaccagttttgccaacattcaagaaaatgccacctgaagcctgcaaaacggggactcgacctcacgttgtgccagtaagtgttagaccacagcacagtcgagaagaagatgagccaaggtcggacaagttgcattctcacgaaatgttgggactgcagacctataatttattctgaataagggttctcaaattcccttttcctgagcacccctttttttttttttgaagatttctgtatttttagttttcaaacatagcaatgttacatattttaaggtatatctgttacaataacaagtgagggcttttttctcaggcatatgaatgactactggacacttctgatttatcctcgttagcagaagtacacaaagcagaaaaggctgaggtctgctatttacacattagtcactgggagcccactctgaaaaagaaacatacttgccaaatggtagcaggctcagtgattaacttaagtgaattcccattgtagtattgttgtatgtatatacatacatgcacacacacacacatatatatatacacgtatacatagatgtatatatgtaatgtatacttaatatatcatacattaaaataatgttctctagttccctgaagtcccttttgaaaccactagttgattataaacctccttaacagttttcagagagtgattccacattatgcatttatccttgttaaaggtttacagtaactgaggttctaatatgacttttataaatactattttacatcttatttttgtctttatttagtaagtaatttataatcactggactgcttaattacctttgaggacaagatggattcatcttatgccagggatttgcatcatgaatttcattaagttatttggcaacctgtaacttgttagtagttcaagtcgaatgtcacccaagtgtgtcatactgtgtttaaatttgtgattttttttaatgaaaattttatcttggaatatttggagatttggggagaaacaaggacaaacacaagagcttaaatttcagaaaatagacagggacctgagggatgctcacggtgagacagctgcgtggtttacactggagatgactcggttgacaggctcgcacaggaagcctcccagttacgggaaagatgaaagtcacatgactgaaacgaaattacccatctcactgtcaggaaactagttcttctttggcatatttctagcaacctttaaaaccatgcttgtttcagtgtcactcagttgtatttctcaagatgtagaagttgatggttttgttggttaatccggtggaaacgggctttgttgtaaaggtaatgaataggaaactcctcagattcaatggttaagaaaatgtgactcccttcacaacctgtaattgccctacaggaaggcaggagtgtttgggtattttttgtatgtttcccacatatgcagagtgtgagagcaggctagtcttagtcccagagtgtgtcacaccgggtatgtgacaatcagacgacgctgtgatccactagatgtgccggggttcattgtgctgtcattgttcctgtcttgatttgaagcacatggttgagggtcattggaagccatcttcatcagtacatgtaaagcttatttacatgtgcaaagtgagtgaagtgacatatttaaactgtgagtagcgactcctcgggtacctttcagtactgtgtgtacaaaccactgcttttggctaagaagctggagagcactttaacaagccagccatctctgttcctgatcagggtctggctctctagaggttgcattagaaatatatttgaaaatgtgccaaagaatttcatcttgtggtcatattaaaaaaatgtacatagttctgaatcctgaggcacatagggttatgtgtgtgcacaagaaaacctgttttttccttatgctttacaataaaggaaataacaaggaaaaaaaaaaaaaaaaaaa

SEQ ID NO: 2 - DNA, 3082 bp, Homo sapiens
cgctcgcggaaaccggaagcggcggctgtccgcggtgccggctgggggcggagaggcggcggtgggctccctggggtgtgtgagcccggtgatggagccgggcccgacagccgcgcagcggaggtgttcgttgccgccgtggctgccgctggggctgctgctgtggtcggggctggccctgggcgcgctccccttcggcagcagtccgcacagggtcttccacgacctcctgtcggagcagcagttgctggaggtggaggacttgtccctgtccctcctgcagggtggagggctggggcctctgtcgctgcccccggacctgccggatctggatcctgagtgccgggagctcctgctggacttcgccaacagcagcgcagagctgacagggtgtctggtgcgcagcgcccggcccgtgcgcctctgtcagacctgctaccccctcttccaacaggtcgtcagcaagatggacaacatcagccgagccgcggggaatacttcagagagtcagagttgtgccagaagtctcttaatggcagatagaatgcaaatagttgtgattctctcagaattttttaataccacatggcaggaggcaaattgtgcaaattgtttaacaaacaacagtgaagaattatcaaacagcacagtatatttccttaatctatttaatcacaccctgacctgctttgaacataaccttcaggggaatgcacatagtcttttacagacaaaaaattattcagaagtatgcaaaaactgccgtgaagcatacaaaactctgagtagtctgtacagtgaaatgcaaaaaatgaatgaacttgagaataaggctgaacctggaacacatttatgcattgatgtggaagatgcaatgaacatcactcgaaaactatggagtcgaactttcaactgttcagtcccttgcagtgacacagtgcctgtaattgctgtttctgtgttcattctctttctacctgttgtcttctaccttagtagctttcttcactcagagcaaaagaaacgcaaactcattctgcccaaacgtctcaagtccagtaccagttttgcaaatattcaggaaaattcaaactgagacctacaaaatggagaattgacatatcacgtgaatgaatggtggaagacacaacttggtttcagaaagaagataaactgtgatttgacaagtcaagctcttaagaaatacaaggacttcagatccatttttaaataagaattttcgatttttctttccttttccacttctttctaacagatttggatatttttaatttccaggcatagcaatgttatctattttaatgtgtatttgtcacaataacagaacatgcaagaacaatcattattttattttataggcatttgattactattctagacttctggtatcttcttactaacataaatatctcaagtagaaaagtttttgaaaactaacatttaaaaattaatcagttacagtaaagactttgaaaaagaaatgtacttgttaggaagtagcttaattaccccccattgcagtattattgttatatatatagttaatatgttgtacatcacaataatatataattcagtctctagtttccctagagtcatttttgaaaccactgattgcaaacctccctgacaatttttaaaagtagtaagccacattacatttatctttgtaaaaagatttatggtaactggtttcttacttgacttttataaatagtattttacatcttatttttgcctttatttcataagtaatttaaaaatcactggattgctttattatattcagggcaatatggattatttttataccaaggatttgcatcgtgaattacattaagttatttggcaatttataatttattactactttaaatcaaatgtagcattatcacactgtatttaaattgtcattttttaaaggaatattttcttcttaagatatatagaggattttggagaagagagacaggaggggtaaaaccagcttaaggttcagcgagcagaaagggacctgagaggatgctcactgtaagactgttggacagtggtgtgtattgaggggatgaatcggaacgatagtctcatgcagaaaatagtgagattaagatcatccttattgtttctaaattatttcaatcagatgaaagtgatacgattgaaatgaaatcacatagttcgtgctcagaaattctattttggtatgtttgtattagcctttagaaaaaacactccgtttcagaattgttcacagttttatttcttaggtttttagagttcaggatttcatttattaatttcttcttgcttttttggtggaaataggctttgttgtaaacattaagaatataaaatctcctctatatagaaacaagaattttgttaaaaagagaatttgaatcccttcctatactataaaatgctctatagggagacaaagtgtttcttttttcttttatgtttactgtttatgtggagtgaaatataaggctcttggatgtataacatactcaaaagctgttacactttctctgatctgctgtgatccactgaaaatgtgctggggtttgttctgctgtcactgtttatgctgctggaacttagcactgtcttgatttgaagcatatgattgagagccatttgaagcaatcttcattaatgcagataaaacaagtttacatgtgcagagttagaaaatgacatgttcaattctgtaagtggtgactttttgagcacctttcagtattatgtatttgtaaaaaccattgtttttggatataaagctaataagcactttaaaaaggaaaaggcagcctttactattttttctggttgagtcattgctctttagacctagcatcagcaatagatttcaaagataagtattaagcgctaccctaaagtgtgtaagtttttcattttgtcatattgaaaaatgatttgcatagtactgaatgttgacacacagcttatatgtatttacaagaatatctttaagtgtttttttgacacattaaaataaaggaaataaggaaattgtaaaaaaaaaaaaaaaaaaaaa

SEQ ID NO: 3 - protein, 338 aa, Mus musculus
MARDAELARSSGWPWRWLPALLLLQLLRWRCALCALPFTSSRHPGFADLLSEQQLLEVQDLTLSLLQGGGLGPLSLLPPDLPDLEPECRELLMDFANSSAELTACMVRSARPVRLCQTCYPLFQQVAIKMDNISRNIGNTSEGPRCGGSLLTADRMQIVLMVSEFFNSTWQEANCANCLTNNGEDLSNNTEDFLSLFNKTLACFEHNLQGHTYSLLPPKNYSEVCRNCKEAYKNLSLLYSQMQKLNGLENKAEPETHLCIDVEDAMNITRKLWSRTFNCSVTCSDTVSVVAVSVFILFLPVVFYLSSFLHSEQKKRKLILPKRLKSSTSFANIQENAT

SEQ ID NO: 4 - protein, 334 aa, Homo sapiens
MEPGPTAAQRRCSLPPWLPLGLLLWSGLALGALPFGSSPHRVFHDLLSEQQLLEVEDLSLSLLQGGGLGPLSLPPDLPDLDPECRELLLDFANSSAELTGCLVRSARPVRLCQTCYPLFQQVVSKMDNISRAAGNTSESQSCARSLLMADRMQIVVILSEFFNTTWQEANCANCLTNNSEELSNSTVYFLNLFNHTLTCFEHNLQGNAHSLLQTKNYSEVCKNCREAYKTLSSLYSEMQKMNELENKAEPGTHLCIDVEDAMNITRKLWSRTFNCSVPCSDTVPVIAVSVFILFLPVVFYLSSFLHSEQKKRKLILPKRLKSSTSFANIQENSN

SEQ ID NO: 5 - DNA, 22 bp, Artificial Sequence
  Description of Artificial Sequence Synthetic primer
ggcgagctatctgttacagtcc

SEQ ID NO: 6 - DNA, 21 bp, Artificial Sequence
  Description of Artificial Sequence Synthetic primer
ttactggcacaacgtgaggtc

SEQ ID NO: 7 - DNA, 21 bp, Artificial Sequence
  Description of Artificial Sequence Synthetic primer
cctgctttgagcataacctgc

SEQ ID NO: 8 - DNA, 19 bp, Artificial Sequence
  Description of Artificial Sequence Synthetic primer
tgacgatatcgctgcgctg

SEQ ID NO: 9 - DNA, 20 bp, Artificial Sequence
  Description of Artificial Sequence Synthetic primer
acatggctggggtgttgaag

SEQ ID NO: 10 - protein, 15 aa, Artificial Sequence
  Description of Artificial Sequence Synthetic peptide
LHSEQKKRKLILPKR

SEQ ID NO: 11 - protein, 15 aa, Artificial Sequence
  Description of Artificial Sequence Synthetic peptide
LNGLENKAEPETHLC

SEQ ID NO: 12 - DNA, 12 bp, Artificial Sequence
  Description of Artificial Sequence Synthetic oligonucleotide
atcggggtgggt

SEQ ID NO: 13 - DNA, 12 bp, Artificial Sequence
  Description of Artificial Sequence Synthetic oligonucleotide
ttgcagaatacc

SEQ ID NO: 14 - DNA, 12 bp, Artificial Sequence
  Description of Artificial Sequence Synthetic oligonucleotide
gcgcaagtgagt

SEQ ID NO: 15 - DNA, 12 bp, Artificial Sequence
  Description of Artificial Sequence Synthetic oligonucleotide
ttacagattgcc

SEQ ID NO: 16 - DNA, 12 bp, Artificial Sequence
  Description of Artificial Sequence Synthetic oligonucleotide
ctgcaggtcagt

SEQ ID NO: 17 - DNA, 12 bp, Artificial Sequence
  Description of Artificial Sequence Synthetic oligonucleotide
ttttaggggcac

SEQ ID NO: 18 - DNA, 12 bp, Artificial Sequence
  Description of Artificial Sequence Synthetic oligonucleotide
gatgcagtgagt

SEQ ID NO: 19 - DNA, 12 bp, Artificial Sequence
  Description of Artificial Sequence Synthetic oligonucleotide
ttctagatgaac

SEQ ID NO: 20 - DNA, 12 bp, Artificial Sequence
  Description of Artificial Sequence Synthetic oligonucleotide
ttctacgtaagt

SEQ ID NO: 21 - DNA, 12 bp, Artificial Sequence
  Description of Artificial Sequence Synthetic oligonucleotide
ccccagccaaac

SEQ ID NO: 22 - DNA, 12 bp, Artificial Sequence
  Description of Artificial Sequence Synthetic oligonucleotide
gcgggggtgggt

SEQ ID NO: 23 - DNA, 12 bp, Artificial Sequence
  Description of Artificial Sequence Synthetic oligonucleotide
ttacagaatact

SEQ ID NO: 24 - DNA, 12 bp, Artificial Sequence
  Description of Artificial Sequence Synthetic oligonucleotide
gtgcaagtaagt

SEQ ID NO: 25 - DNA, 12 bp, Artificial Sequence
  Description of Artificial Sequence Synthetic oligonucleotide
tgacagattgtt

SEQ ID NO: 26 - DNA, 12 bp, Artificial Sequence
  Description of Artificial Sequence Synthetic oligonucleotide
cttcaggtattt

SEQ ID NO: 27 - DNA, 12 bp, Artificial Sequence
  Description of Artificial Sequence Synthetic oligonucleotide
ttttaggggaat

SEQ ID NO: 28 - DNA, 12 bp, Artificial Sequence
  Description of Artificial Sequence Synthetic oligonucleotide
gatgcagtaagt

SEQ ID NO: 29 - DNA, 12 bp, Artificial Sequence
  Description of Artificial Sequence Synthetic oligonucleotide
ttccagatgaac

SEQ ID NO: 30 - DNA, 12 bp, Artificial Sequence
  Description of Artificial Sequence Synthetic oligonucleotide
ttctgcgtaagt

SEQ ID NO: 31 - DNA, 12 bp, Artificial Sequence
  Description of Artificial Sequence Synthetic oligonucleotide
atctagccaaac